(19) United States
(12) Patent Application Publication
(10) Pub. No.: US 2026/0048272 A1
(43) Pub. Date: Feb. 19, 2026

(54) APPARATUS AND METHOD FOR OPTICAL SKIN TREATMENT AND IN-TREATMENT MONITORING THEREOF

(71) Applicant: LUMENIS BE LTD., YOKNEAM (IL)

(72) Inventors: Andrey Gandman; Israel Schuster

(21) Appl. No.: 19/100,075

(22) PCT Filed: Jul. 30, 2023
(86) PCT No.: PCT/IL2023/050785

§ 371 Date: Jan. 30, 2025

Related U.S. Application Data
(63) Continuation-in-part of application No. 17/226,235, filed on Apr. 9, 2021.
(63) Continuation-in-part of application No. 17/565,709, filed on Dec. 30, 2021, now abandoned.
(60) Provisional application No. 63/132,554, filed on Dec. 31, 2020.
(60) Provisional application No. 63/393,932, filed on Jul. 31, 2022.
(60) Provisional application No. 63/425,493, filed on Nov. 15, 2022.

(51) Int. Cl. A61N 5/06; A61N 5/073

(52) U.S. Cl. CPC A61N 5/0616; A61N 2005/0626; A61N 2005/063; A61N 2005/0665; A61N 2005/073

(57) ABSTRACT

An apparatus and method for optical skin treatment procedures and in-treatment monitoring thereof. The apparatus is operable in two modes: a treatment mode for delivery of treatment light to an area of a patient's skin abutted to a light guide of the apparatus; and a diagnostic mode for acquiring one or more images of the abutted area of skin. Switching between the two modes may be made in a short time, facilitating monitoring during treatment. In exemplary embodiments, the apparatus sends image data to an analyzer, which analyzes images and computes an optimal treatment course, at least optimal parameters (intensity, pulse width, repetition rate) for the next delivery, and sends the optimal treatment course to the controller for implementation by the apparatus in real time.

### FIELD OF THE INVENTION

The invention is in the field of optical skin treatment and diagnostics, and in particular related to an apparatus and method for optical skin treatment and in-treatment monitoring thereof.

### BACKGROUND TO THE INVENTION

Treating tissue with light such as laser and intense pulsed light (IPL), for cosmetic or therapeutic purposes is known. For example, U.S. patent application Ser. No. 17/226,235, assigned to the assignee of the present disclosure and incorporated herein by reference, discloses a method and apparatus for real time monitoring of cosmetic laser aesthetic skin treatment procedures. U.S. patent application Ser. No. 17/203,994, assigned to the assignee of the present disclosure and incorporated herein by reference, discloses a method and system for determining an optimal set of operating parameters for an aesthetic skin treatment unit. U.S. patent application Ser. No. 17/565,709, assigned to the assignee of the present disclosure and incorporated herein by reference, discloses a method and system for real time monitoring of laser aesthetic skin treatment procedures. U.S. Pat. No. 6,280,438 B1, assigned to the assignee of the present disclosure and incorporated herein by reference, discloses a method and apparatus for electromagnetic treatment of the skin, including hair depilation. US Patent Application Publication 2020/0188687 A1, assigned to the assignee of the present disclosure and incorporated herein by reference, discloses selective skin treatments utilizing laser-equivalent intense pulsed light devices.

### SUMMARY

The presently disclosed subject matter provides apparatuses and methods for optical diagnosis and treatment of various conditions of tissue. The disclosed apparatuses enable fast switching between diagnostic and treatment modes during a treatment session to optimize treatment in real-time.

In accordance with a first aspect of the present disclosure, there is provided an apparatus for optical skin treatment and in-treatment monitoring thereof, the apparatus comprising:
a controller;
a light guide comprising a contact surface configured for abutment to an area of skin;
an irradiator assembly configured, upon command of the controller, to provide treatment light; and
an illuminator assembly configured, upon command of the controller, to provide illumination light;
an imaging assembly configured, upon command of the controller, to receive reflected or backscattered illumination light and generate skin image output data; and
an optical path selection assembly, configured to establish, upon command of the controller, a selected mode of the apparatus comprising either:
a treatment mode enabling the treatment light from the irradiator assembly to enter the light guide and irradiate the abutted area of skin to provide the skin treatment; or
a diagnostic mode, enabling the illumination light from the illuminator assembly to enter the light guide and illuminate the area of skin abutted to the contact surface; and the reflected or backscattered illumination light to traverse the light guide towards an image sensor of the imaging assembly to provide the in-treatment monitoring.

In some embodiments, the apparatus further comprising an optics assembly configured to collect the reflected or backscattered illumination light and direct it towards the imaging assembly.

In some embodiments, the irradiator assembly comprises an extended light source and one or more reflectors between the extended light source and the light guide, the reflector being configured to capture and reflect rays of the extended light source towards the light guide.

In some embodiments, the skin image is of an area of skin abutting a portion of an area of the contact surface.

In some embodiments, the illuminator assembly comprises an illumination polarizer configured so that said illumination light is P-polarized in a plane of incidence of the illumination light.

In some embodiments, the imaging assembly comprises a detection polarizer, disposed upstream the image sensor and arranged cross-polarized with the illumination polarizer.

In some embodiments, one or more of the illumination and detection polarizers is/are grid wire polarizers.

In some embodiments, the illuminator assembly comprises one or more sets of one or more light sources, each set having a different illumination spectrum, and the controller is further configured to select illumination from one or more of the sets of light sources.

In some embodiments, the sets of light sources comprise LEDs. The illuminator assembly may further comprise an optical sensor configured for monitoring illumination intensities of the LEDs.

In some embodiments, the optics assembly comprises a focus adjustment mechanism to focus the reflected or backscattered illumination light on the image sensor of the imaging assembly.

In some embodiments, the imaging assembly comprises a position adjustment mechanism to adjust position of the image sensor relative to a plane of the image sensor.

In some embodiments, the optical path selection assembly comprises a translatable platform, translatable on a stationary frame of the apparatus between two end points of travel to establish said treatment and diagnostic modes respectively, wherein the irradiator assembly and the light guide are in rigid connection with the stationary frame. The optics assembly may comprise a first folding mirror, a lens assembly, and a second folding mirror, wherein at least the illuminator assembly and the first folding mirror are rigidly affixed to the translatable platform. The lens assembly and second folding mirror may be further rigidly affixed to the translatable platform. The imaging assembly may be further rigidly affixed to the translatable platform. The image sensor may be arranged inline with the lens assembly; and the lens assembly is rigidly affixed to the translatable platform. The apparatus may comprise a limit switch at either or each of the end points of travel of the translatable platform, configured to decelerate the translatable platform to a stop at the end points of travel. A drive motor of the translatable platform may be configured for calibration of positions for the imaging path selection and/or the treatment path selection.

In some embodiments, the optical path selection assembly comprises a pair of movable mirrors configured to be moved between at least two positions to respectively establish said treatment and diagnostic modes.

In some embodiments, the optical path selection assembly comprises a single movable mirror configured to be moved between at least two positions to respectively establish said treatment and diagnostic modes.

In some embodiments, the optical path selection assembly comprises two light input surfaces configured to respectively establish said treatment and diagnostic modes. The two input surfaces may be integral with a surface of the light guide.

In some embodiments, the apparatus further comprises an image compositor, enabled to acquire pixel data of the images taken under one or more of the different illumination spectra and apply linear mixing and/or algorithms to produce one or more composite images. The image compositor may be configured to produce one or more of the following types of composite images: an RGB image, a melanin map, an erythema level map, a blood vessel map, a photon scattering map, an intermediate melanin map; a deep melanin map, and a blood vessel depth map.

In accordance with a second aspect of the present disclosure, there is provided a method for optical imaging and treatment of skin, the method comprising the following steps:
a. abutting a contact surface of a light guide of an apparatus, for optical skin treatment and in-treatment monitoring thereof, to an area of skin of a patient;
b. commanding, by a controller of the apparatus, an optical path selection assembly of the apparatus to place the apparatus in a diagnostic mode, whereby illumination and imaging paths are established;
c. commanding, by the controller, an illuminator assembly of the apparatus to illuminate the area of skin abutted to the contact surface;
d. collecting, by an optics assembly of the apparatus, backscattered illumination light from the abutted area of skin;
e. focusing, by the optical assembly, an image of the abutted area of skin on an image sensor of the apparatus;
f. commanding, by the controller, collection of image data of the image from the image sensor;
g. commanding, by the controller, the optical path selection assembly to place the apparatus in a treatment mode, whereby a treatment light delivery path is established; and
h. commanding, by the controller, an irradiator assembly of the apparatus to generate treatment light having parameters based on the analysis of the image data, and directing the treatment light through the light guide to irradiate the area of skin with the treatment light.

In some embodiments, the method of further comprises:
i. acquiring pixel data from the apparatus of images taken under one or more illumination spectra; and
j. applying linear mixing and/or algorithms to the acquired images to produce one or more composite images to be analyzed for determining parameters of the treatment light.

### BRIEF DESCRIPTION OF THE DRAWINGS

FIGS. 1A and 1B are functional block diagrams of an apparatus for optical cosmetic skin treatment procedures and in-treatment monitoring thereof, according to some exemplary embodiments of the invention;

FIGS. 2A and 2B depict an apparatus for Intense Pulsed Light (IPL) skin treatment procedures and in-treatment monitoring thereof, according to some exemplary embodiments of the invention;

FIGS. 3A and 3B are cross-sectional drawings of an apparatus for IPL skin treatment procedures and in-treatment monitoring thereof, according to some exemplary embodiments of the invention;

FIG. 4 depicts a printed circuit board (PCB) of an illuminator assembly, according to some exemplary embodiments of the invention;

FIG. 5 depicts an illuminator assembly, a light guide, an optics assembly, and an image sensor, as well as an optical ray trace of light reflected/backscattered through the light guide, according to some embodiments of the invention;

FIGS. 6A-6C depict a non-limiting exemplary simulation of background images from LED illumination sources;

FIG. 7 depicts a translating platform of the apparatus, forming a non-limiting example of the optical path selection assembly, according to some exemplary embodiments of the invention;

FIG. 8 depicts a random ray-trace simulation of treatment light through a reflector and light guide;

FIGS. 9A and 9B depict another non-limiting example of the optical path selection assembly of the apparatus, in accordance with some exemplary embodiments of the invention;

FIGS. 10A and 10B depict another non-limiting example of the optical path selection assembly of the apparatus, in accordance with some exemplary embodiments of the invention;

FIG. 11 depicts an apparatus having another non-limiting example of the optical path selection assembly, in accordance with some exemplary embodiments of the invention;

FIGS. 12A and 12B depict the apparatus mounted in one of two half-shells of a handpiece and a fully assembled handpiece, respectively, according to some exemplary embodiments of the invention;

FIG. 13 depicts a functional block diagram of a system for skin imaging and analysis, according to some exemplary embodiments of the invention; and

FIG. 14 depicts a method for optical skin treatment procedures and in-treatment monitoring thereof, according to some embodiments of the invention.

### DETAILED DESCRIPTION

An aspect of the invention relates to an apparatus and method for optical skin treatment procedures and real-time monitoring thereof. In some embodiments, the optical skin treatment is for cosmetic purposes. In some embodiments, the optical skin treatment is for dermatological, therapeutic purposes. It is noted that real-time or in-treatment monitoring, as used herein, encompasses before-, during-and/or after-treatment monitoring possibilities.

In some embodiments, the apparatus is operable in two modes: a treatment mode for delivery of treatment light from, e.g. an intense pulsed light (IPL) source, to an area of a patient's skin abutted to a light guide of the apparatus; and a diagnostic mode for acquiring an image of the abutted area of skin. In some embodiments, the apparatus is a handpiece of a therapeutic IPL system, having a tethered connection to the system. The switching of the apparatus between the two modes may be made in a relatively short time (at most a few seconds, in some embodiments), such that in-treatment monitoring is achievable. Furthermore, in exemplary embodiments, the apparatus sends image data to an analyzer, which analyzes images and computes an optimal treatment course, at least the optimal parameters (intensity, pulse width, repetition rate) for the next delivery, and sends the optimal treatment course to the controller of the apparatus in real time. The apparatus enables, for example, iterations of imaging the skin after a delivery of treatment light and deciding parameters of the next delivery without undue delay.

For treatment using an IPL source (See U.S. Pat. No. 6,280,438 B1 and US Patent Application Publication 2020/0188687 A1, both assigned to the assignee of the present invention and incorporated herein by reference), a light guide is necessitated by the uncollimated nature of light emission from the IPL source. The light guide serves to contain the uncollimated treatment light rays within a defined area of skin. Much of the energy in the treatment light rays undergoes one or multiple reflections within the light guide.

In the case of laser treatment (see U.S. patent application Ser. No. 17/565,709, assigned to the assignee of the present invention and incorporated herein by reference), in-treatment monitoring is facilitated by air gaps from the laser source to the skin and from the skin to the imaging system. In contrast, an IPL source requires the presence of a light guide between the IPL source and the skin. The light guide also appears in the path from the skin (abutted to the light guide) to the imaging system in the handpiece, interrupting a continuous imaging path in a single medium (i.e. air) and thereby hindering the capturing of skin images. The invention offers a solution, as further described herein.

“In-treatment” monitoring does not imply that monitoring is necessarily taking place at exactly the same time as treatment. The apparatus may switch between the treatment mode and the diagnostic mode within a period of time sufficiently short to an operator, i.e. several seconds. During a treatment of a single patch of skin abutted to the light guide, the apparatus may switch between treatment and diagnostic modes multiple times.

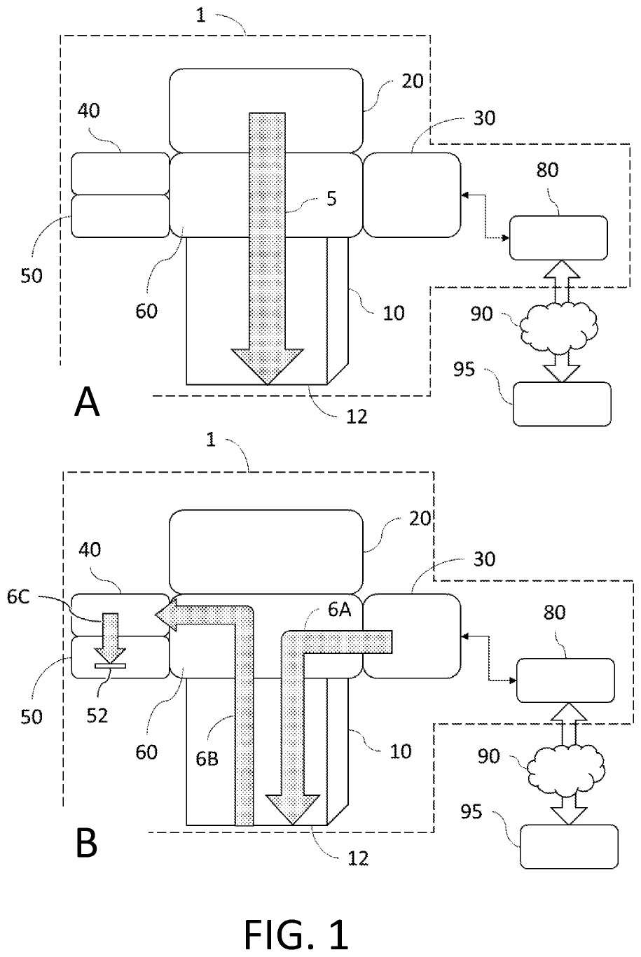

Reference is now made to FIG. 1A-1B, depicting functional block diagrams of an apparatus 1 for optical skin treatment and in-treatment monitoring thereof, according to some non-limiting embodiments. FIG. 1A depicts operation of the apparatus 1 in a treatment mode and FIG. 1B depicts operation of the apparatus 1 in a diagnostic mode. The functional diagrams in FIGS. 1A and 1B are given for schematic purposes only; no inference of relative size or geometry (of elements or optical paths) is to be drawn from the figures.

As shown in the figures, the apparatus 1 comprises the following elements: a light guide 10 with a contact surface 12 configured to be abutted to an area of skin; an irradiator assembly 20 providing treatment light to the abutted area of skin; an illuminator assembly 30 providing illumination light to the abutted area of skin; an optics assembly 40 and an image sensor assembly 50, for acquiring an image of illumination light backscattered and/or reflected from the abutted area of skin; and an optical path selection assembly 60, configured to establish the apparatus in either a treatment mode or a diagnostic mode. Selection of treatment mode or diagnostic mode may be made by the operator, either mechanically (for example, via a switch) or by electronic command through a processor or controller (hereinafter controller) 80 of the apparatus 1. In some embodiments, the irradiator assembly 20 is connected to or is housed in a therapeutic IPL source system, by a tethered connection to the IPL source system (not shown).

In some embodiments, when the apparatus 1 is in the treatment mode (FIG. 1A), treatment light 5 from the irradiator assembly 20 enters the light guide 10, exits the light guide 10 through the contact surface 12, and irradiates the area of skin abutted to the contact surface 12, thereby treating the irradiated area of skin.

In some embodiments, when the apparatus 1 is in the diagnostic mode (FIG. 1B), illumination light 6A from the illuminator assembly 30 enters the light guide 10, exits the light guide 10 through the contact surface 12, and illuminates the area of skin abutted to the contact surface 12. Additionally, in the diagnostic mode, reflected and/or backscattered light 6B from the abutted area of skin enters the light guide through the contact surface 12, exits the light guide 10, and is collected and focused by the optics assembly 40. The focused light 6C enters the image sensor assembly 50 and is received by an image sensor therein, onto which the focused light 6C forms an image of the abutted area of skin.

The optical path selection assembly 60 may employ any combination of optical, opto-mechanical, mechanical, electro-/magneto-optical, electrical, and electronic means to establish a treatment mode or diagnostic mode.

The apparatus 1 may further comprise a controller 80 (which can be configured as one or more distributed controllers each responsible for controlling a different part/function of the apparatus), whose functions include one or more of the following: control of the irradiator assembly 20 (e.g., repetition rate, treatment light output level, and/or cooling); control of the illuminator assembly 30 (e.g., illumination output light level/parameters); control of the image sensor 52 (e.g., acquisition rate and acquisition time), as well as receiving image pixel data therefrom; selection of treatment mode or diagnostic mode of the optical path selection assembly 60; cooling control of the irradiator assembly and/or the image sensor 52; or any combination thereof.

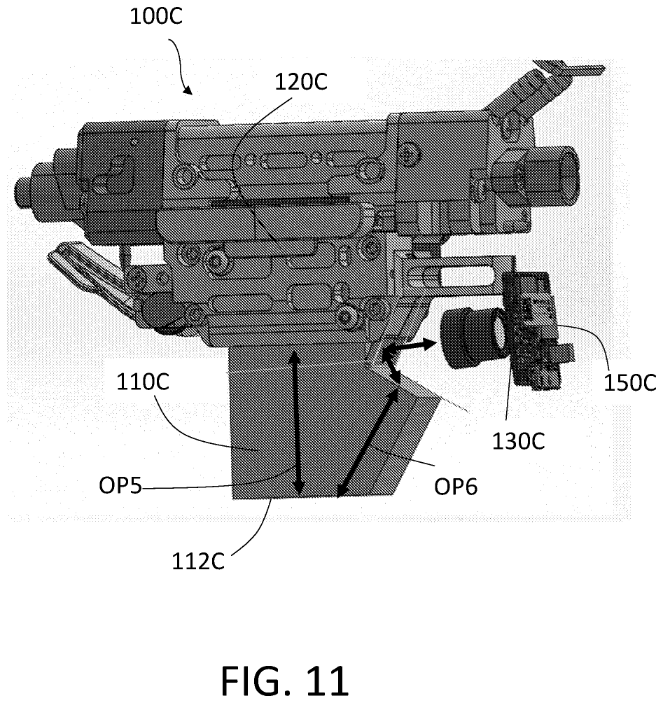

In some embodiments, the controller 80 may be in communicative connection with an analyzer 95 which can form part of the apparatus or be external thereto. In the latter case, the connection may be over a network 90 and the analyzer 95 may be part of a cloud service that services multiple units of the apparatus 1 at different locations. The controller 80 may send image data (pixel data and/or processed image data) of images received by the image sensor 52 while the apparatus 1 is in diagnostic mode, to the analyzer 95. The analyzer 95 may also receive metadata, such as patient data, past conditions of the patient under treatment, etc. In some embodiments, the analyzer 95 analyzes the received data and determines an appropriate treatment regimen; at least for the treatment of the next iteration cycle, when the apparatus 1 will have switched into treatment mode. Detailed teachings about the analyzer 95, including machine learning methods thereof, may be found in U.S. patent application Ser. No. 17/203,994, assigned to the assignee of the present invention and incorporated herein by reference. In some embodiments, the controller 80 is further connected to at least one input/output (I/O) utility (not shown) for the use of an operator, and to a display (See FIG. 11) to present the images of the tissue under treatment.

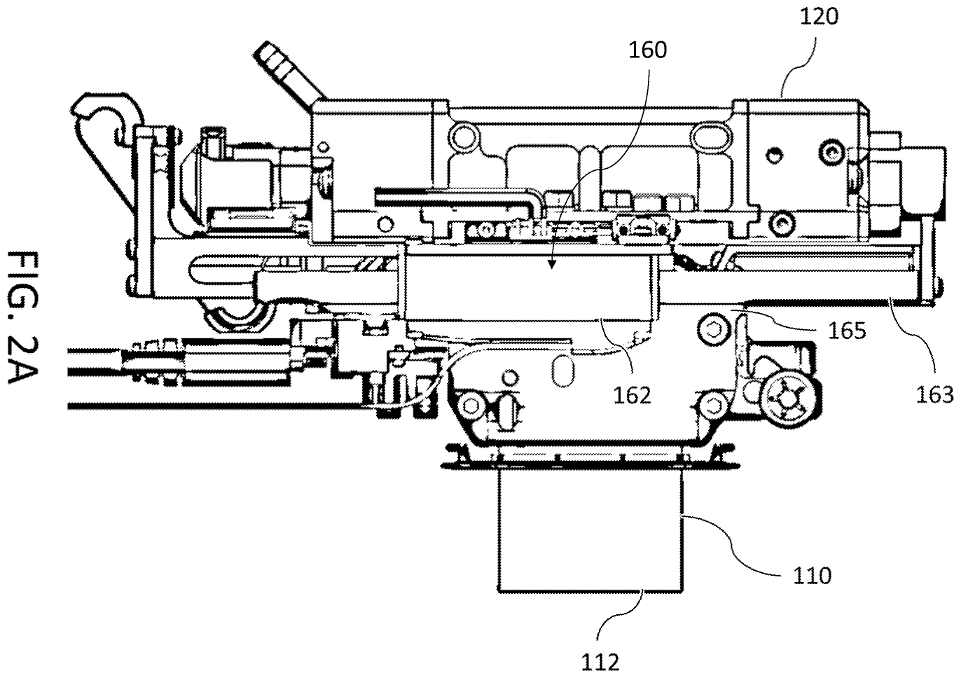

Reference is now made to FIGS. 2A-2B, depicting a non-limiting example of an apparatus 100 for IPL skin treatment and in-treatment monitoring thereof, according to some embodiments. In FIG. 2A, the apparatus 100 is in a treatment mode. In FIG. 2B, the apparatus is in a diagnostic mode.

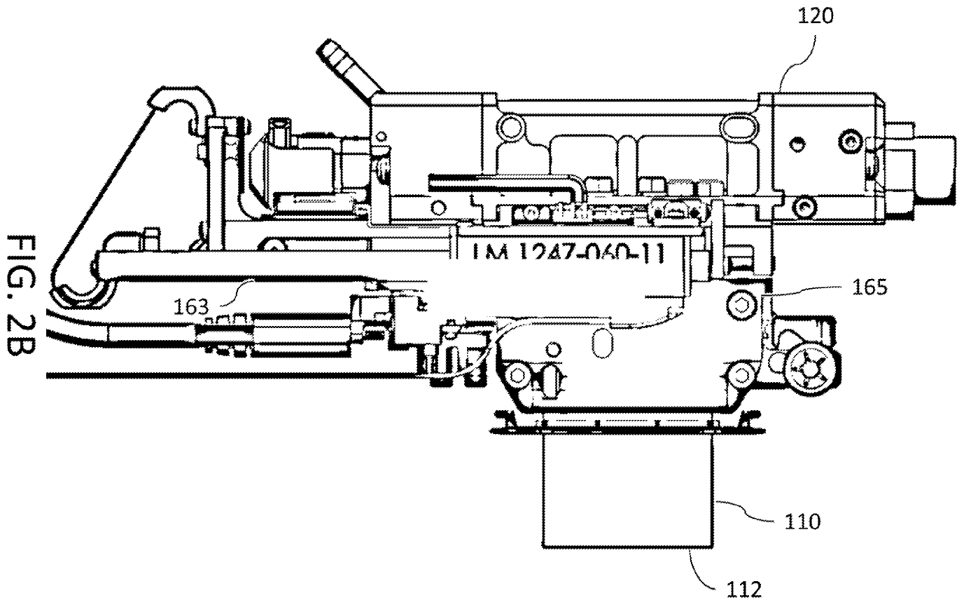

Reference is further made to FIGS. 3A-3B, depicting cross-sectional views of the apparatus 100 for IPL skin treatment and in-treatment monitoring thereof, according to some embodiments, and showing some elements obscured in FIGS. 2A-2B. In FIG. 3A, the apparatus 100 is in a treatment mode. In FIG. 3B the apparatus is in a diagnostic mode. Note further that some features appearing in FIG. 3B may be obscured in FIG. 3A, and vice versa.

As illustrated, the apparatus 100 comprises a light guide 110, an irradiator assembly 120; an illuminator assembly 130; an optics assembly 140; an image sensor assembly 150; and an optical path selection assembly 160.

The light guide 110 may have a contact surface 112 that is abutted to an area of skin of a patient during treatment and diagnostics. The light guide 110 may be shaped as a rectangular polyhedron with a flat contact surface 112, as shown. Alternatively, the light guide 110 may be of any suitable shape and have a contact surface 112 of any suitable shape. For example, the contact surface 112 may be shaped for matching a particular part of the skin to be treated. Moreover, the light guide 110 and contact surface 112 may be sized according to requirements of the particular part of the skin.

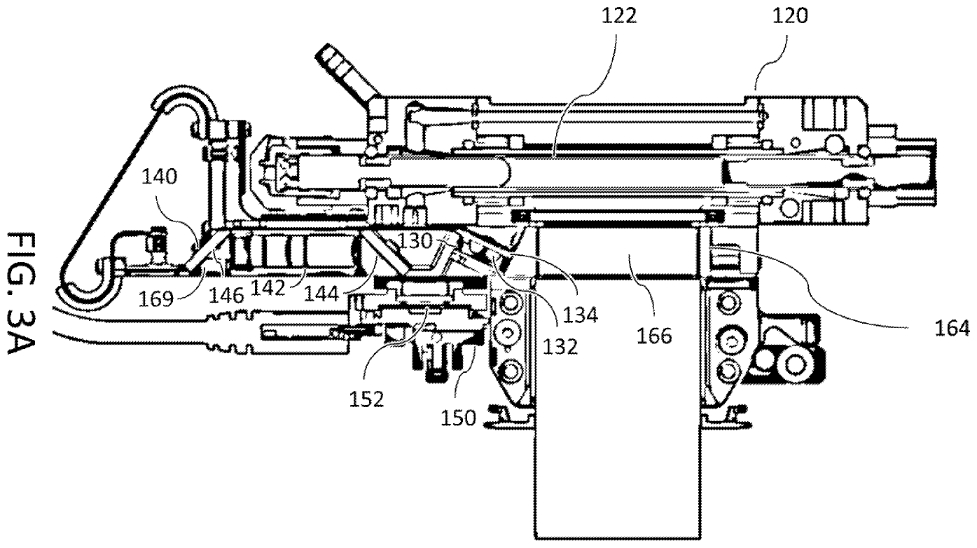

The irradiator assembly 120 comprises a treatment light source 122 that provides treatment light, upon an appropriate instruction by an operator. The treatment light passes through the light guide 110, exits the light guide 110 through the contact surface 112, and treats the area of skin abutted to the contact surface 112. The treatment light source 122 may be a Xe flashlamp, as shown, or may be any light source with characteristics (e.g. spectrum and intensity) suitable for the required skin treatment. The light source can be a point light source, a non-collimated extended light source, or can be a laser or any light source suited for skin treatment. The controller (not shown) activates the treatment light source 122 while the apparatus 100 is in treatment mode, as shown in FIGS. 2A and 3A and further described herein. The illuminator assembly 130 comprises one or more illumination light sources 132. The illumination light sources 132 provide illumination light for imaging the abutted area of skin. The illumination light passes through the light guide 110, exits the light guide 110 through the contact surface 112, and illuminates the area of skin abutted to the contact surface 112. The illuminator assembly 130, in some embodiments, comprises a printed circuit-board (PCB) 134 with one or more LEDs 132 mounted thereon (further described herein with reference to FIG. 4). However, the illumination light sources may be any light source with characteristics (e.g. spectrum and intensity) suitable as illumination for diagnostic imaging of skin. The controller may activate the illuminator assembly 130 while the apparatus 100 is in diagnostic mode, as shown in FIGS. 2B and 3B and further described herein.

The abutted area of skin will reflect and backscatter the illumination light from the illuminator assembly. In this disclosure, “reflected light” refers to specular reflections at the interface of the light guide and the skin; and “backscattered light” refers to light that penetrates the light guide-skin interface and is scattered by tissue at some internal depth of the skin beyond the light guide-skin interface.

The treatment light and illumination light may be delivered through a single input surface (as shown in FIGS. 2A-3B for example) or through different input surfaces (as shown in FIG. 11 for example), of the light guide.

The optics assembly 140 collects and focuses reflected and/or backscattered light from the area of skin abutted to the contact surface 112. The optics assembly 140 may refract and/or reflect rays of skin-reflected/backscattered illumination light on the image sensor assembly 150, forming an image of the abutted area of skin (hereinafter, “skin image”) thereon. In some embodiments, the optics assembly 140 comprises one or more focusing elements (lenses and/or curved mirrors) and may comprise one or more folding mirrors. In the described example, the focusing optics assembly 140 comprises a lens assembly 142 and two folding mirrors 144, 146. The lens assembly 142 may comprise a focus adjustment mechanism. Additionally or alternatively, the image sensor assembly 150 may comprise a position adjustment mechanism for adjusting the position of the image sensor 152 perpendicular to the plane of the image sensor 152, in order to focus the image on the plane of the image sensor 152. The focus adjustment mechanism of the lens assembly 142 and the position adjustment mechanism of the image sensor assembly 150 may be manual, automatic, or any combination thereof.

In some embodiments, the optical path selection assembly 160 comprises an actuator such as a motor 162 with a driving rod 163 (visible in FIGS. 2A and 2B), and a translatable platform 164 (visible in FIGS. 3A and 3B). The driving rod 163 is rigidly affixed to the translatable platform 164. The motor 162, rigidly affixed to the apparatus frame 165, is controllable to translate the driving rod 163. The motor 162 thereby translates the translatable platform 164 linearly in two opposite directions.

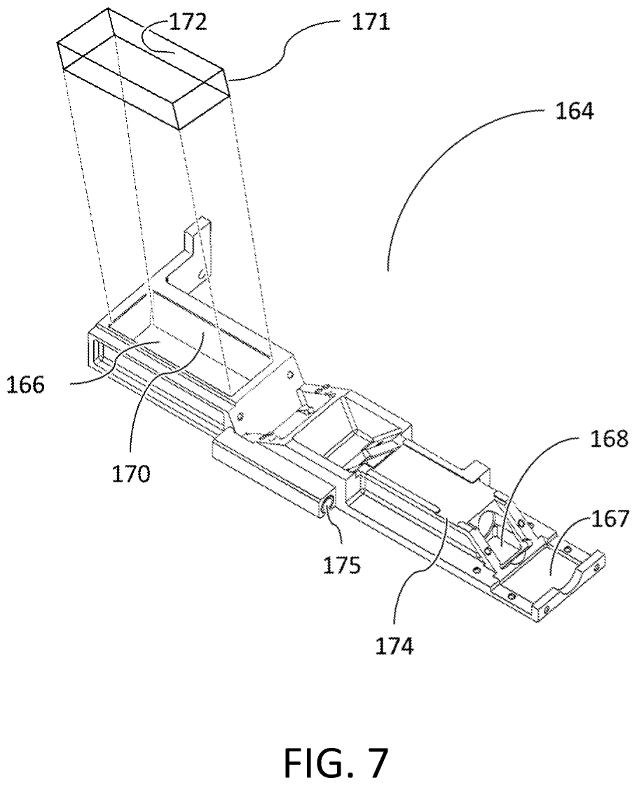

The optical path selection assembly 160 may further comprise a stationary parallel rod (not shown), rigidly connected to the frame 165 of the apparatus 100 (on the side of the apparatus opposite the motor 162 and driving rod 163). The translatable platform 164 travels along the parallel rod through a parallel bearing 175 (visible in FIG. 8) rigidly affixed to the translatable platform 164. The parallel rod and parallel bearing 175, in conjunction with the motor 162 and driving rod 163, provide support and translational stability to the translatable platform 164.

The optical path selection assembly 160 may further comprise a limit switch (not shown) at either or both ends of travel of the translatable platform 164. Upon receiving a limit signal, the controller and/or motor 162 are configured to decelerate the translatable platform 164 to a stop. Either or both limit switches may be disposed corresponding to the desired stop position of the translatable platform 164, at which the translatable platform 164 is stationarily positioned for the treatment mode or diagnostic mode of the apparatus 100. A calibration procedure of the motor 162 may be implemented for the controller to determine how many encoder counts to operate the motor 162 after receiving a limit switch signal, in order for the translatable platform 164 to be positioned for optimal alignment.

In some embodiments, the illuminator assembly 130 and the optics assembly 140 are rigidly affixed to the translatable platform 164. The irradiator assembly 120 is in rigid connection with the stationary frame 165 of the apparatus 100. The image sensor assembly 150 is also rigidly connected to the stationary frame 165, rather than to the translatable platform 164, thereby minimizing the weight carried by the translatable platform 164.

In some embodiments, when the apparatus 100 is in the treatment mode, a delivery opening 166 (visible in FIG. 3A) is disposed between the irradiator assembly 120 and the light guide 110, enabling treatment light from the irradiator assembly to enter the light guide 110 towards the tissue.

In some embodiments, when the apparatus 100 is in the diagnostic mode, the illuminator assembly 130 with an illumination opening 167, and a first folding mirror 144 with a collection opening 168 (visible in FIG. 3B), are disposed near the light guide. The illumination light is thereby enabled to pass through the illumination opening 167 and enter the light guide 110 and reflected/backscattered light is thereby enabled to exit the light guide through the collection opening 168 and reach the first folding mirror 144 and then the lens assembly 142 positioned after the folding mirror 144.

Additionally, with the apparatus 100 in the diagnostic mode, an imaging opening 169, is disposed between the second folding mirror 146, located after the lens assembly, and the image sensor assembly 150, thereby enabling light exiting the lens assembly 142 and folded by the second folding mirror 146 to pass through the imaging opening 169 and reach the image sensor 152.

Other configurations may be apparent to skilled persons, based on this disclosure, to establish a selected optical path of either a skin imaging path or a treatment-light delivery path. Such configurations include both opto-mechanical and non-mechanical means (e.g. electro-optic switching).

As an example, it is possible in some embodiments for the apparatus 100 to translate only the first folding mirror 144 of the optics assembly 140 during optical path selection, with the lens assembly 142 and the second folding mirror 146 rigidly affixed to the frame 165.

As another example, in some embodiments there is an unfolded optical path between the light guide 110 and the image sensor 152, without a need for folding mirrors; with the lens assembly 144 and the image sensor assembly 150 are in line and in rigid connection with the translatable platform 164. An advantage of folding the optical path with folding mirrors 144, 146, in comparison with an unfolded path, is a minimized gap (e.g. through the delivery opening 166) between the treatment light source 122 and the light guide 110, thereby minimizing losses in treatment light.

While in the above-described examples, the optical path selection assembly is configured to align the optics assembly with the light guide and enable the diagnostic mode, and misalign the optics assembly with the light guide to enable the treatment mode, it can be appreciated that other configurations are available. For example, it could be that the illumination light should be folded on the path between the illuminator assembly to the light guide and thus the optical path selection assembly will replace the optics assembly affiliated with the imaging assembly with another optics assembly affiliated with the illuminator assembly.

In some embodiments, the apparatus for both treatment and diagnostic modes may have detachable parts or various configurations of the image sensor optics and illuminator assembly such that an optical path from a treatment source is unobstructed and image data can be captured.

Reference is now made to FIG. 4, depicting a non-limiting example of an illuminator assembly 130 in a form of a PCB 134, according to some embodiments. LEDs 132 may be arranged in sets on the PCB 134, each LED set characterized by an optical spectral output. The penetration depth in the skin of illumination light is wavelength dependent; LEDs of multiple optical spectral outputs may be illuminated serially, at different times, and images under each illumination spectrum taken, to achieve a composite skin image of layers of interest. Using serial spectral illumination may obviate a color-sensing capability of the image sensor 152. In some embodiments, there are LEDs 132 whose peak-wavelength emissions are nominally 450, 490, 570, 590, 660, 730, and 860 nm. In some embodiments, there are two or more LEDs 132 for each of some wavelengths, which may enable providing spatially symmetric illumination on the area of skin abutted to the contact surface 112 of the light guide 110. For example, as shown in the figure, there may be two of each of the 570, 590, 730 nm LEDs 132.

Optionally, the PCB 134 further comprises an optical sensor, which can be a photodiode 136. The photodiode 136 measures illumination light that is reflected or backscattered to the illuminator assembly 130, enabling monitoring of changes in output illumination of LEDs 132. The controller, or a dedicated closed-loop circuit, may monitor the output of the photodiode 136, in order to stabilize the output illumination of one or more of the LEDs 132.

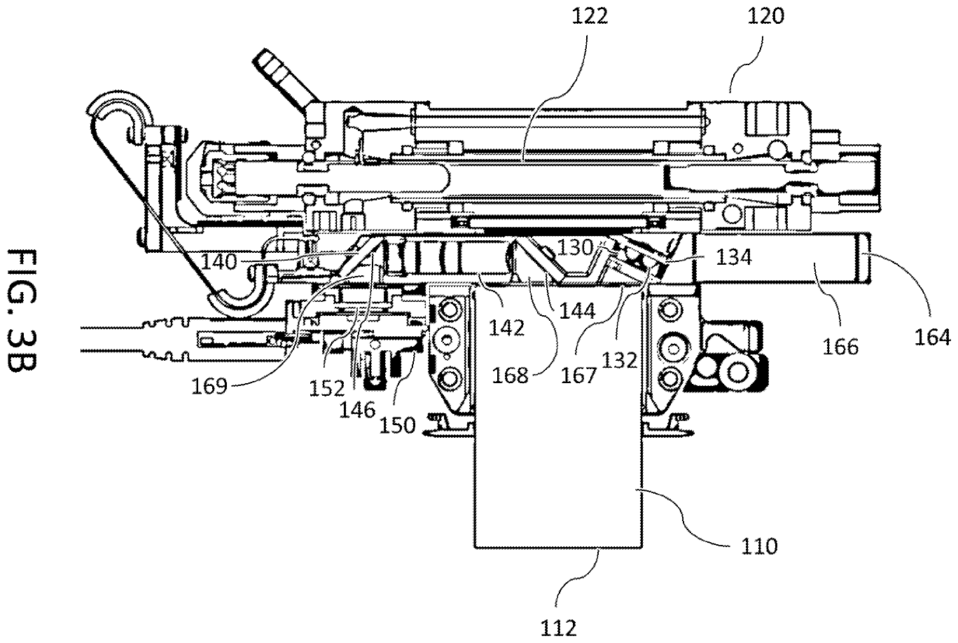

Reference is now made to FIG. 5, depicting optical ray traces of illumination light originated from the illuminator assembly 130, backscattered/reflected from the abutted area of skin, entering and traversing the light guide 110, collected by the optics assembly 140, and focused by the lens assembly 144 onto the image sensor 152, according some embodiments of the invention.

An illumination polarizer 138 may be positioned between the illumination light sources 132 and the light guide 110. The illumination polarizer 138 may be arranged so that illumination light on the contact surface 112 and abutted area of skin is P-polarized in the plane of incidence of the illumination light, minimizing internal reflections from the contact surface-skin interface, thereby minimizing loss of illumination light not reaching internal layers of the skin as well as minimizing the reflections that enter the optics assembly 140 and reach the image sensor 152 as stray light. In some embodiments, the illumination polarizer 138 is mounted in the illuminator assembly 130, thereby preventing unpolarized stray light from exiting the illuminator assembly 130 and reaching the image sensor 152.

A detection/sensor polarizer 154 may be positioned between the light guide and the image sensor 152. In some embodiments, the sensor polarizer is in proximity to the image sensor 152 and part of the image sensor assembly 150. The sensor polarizer 154 may be arranged cross-polarized with the illumination polarizer 138. The reason for cross polarization of the sensor polarizer 154 with the illumination polarizer 138 is to block light reflected specularly by the skin from reaching the image sensor 152 (since the reflections retain incident polarization while backscattering of the illumination light randomizes polarization). Thus, the crossed polarizers 138, 154 cause the image sensor 152 to receive a skin image of internal layers of skin and suppress a skin image of the surface of the skin.

Either or both of the illumination polarizer 138 and the sensor polarizer 154 are optionally grid wire polarizers.

The skin image may be of an area of skin abutting a portion 112A of the contact surface 112. Including the area of skin outside of the portion 112A in the image may result in imaging the illumination sources 132 themselves. (Note that the center of the portion 112A is offset from the center of the contact surface 112). The extent of the imaged portion 112A may be limited by the field-of-view of the optics assembly 140.

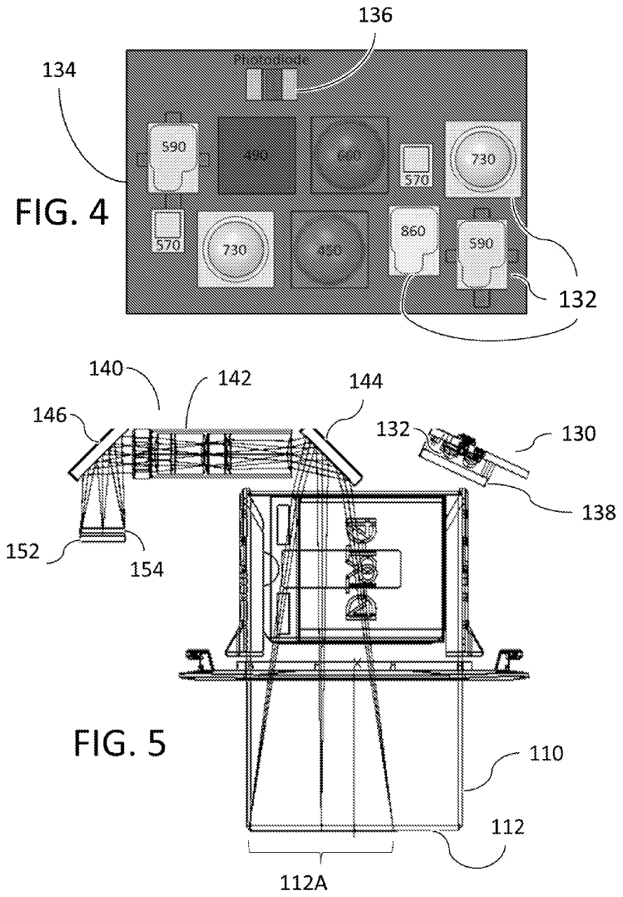

FIGS. 6A-C depict simulated background images, with illumination sources 132 lit and the contact surface 112 positioned against a dark background. FIG. 6A shows the background image with the illuminator assembly 130 positioned as shown in FIG. 5. FIGS. 6B-C show background images when the illuminator assembly is shifted 0.2 mm to the left. FIG. 6B show background images for the 660 LED and FIG. 6C for the 730 nm LEDs. The presence of the background LED images in FIGS. 6B and 6C illustrates the difficulties encountered in trying to illuminate the skin through the light guide and why only a portion of the skin abutted to the light guide 110 may be imaged.

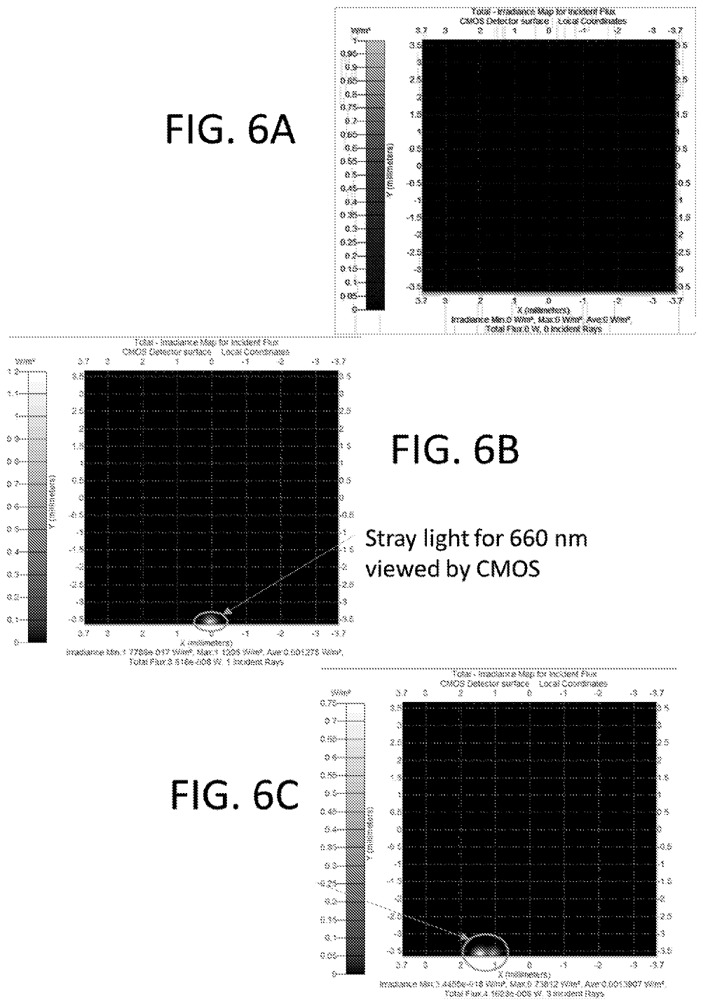

Reference is now made to FIG. 7, depicting a translatable platform 164 of the apparatus 100, according to some embodiments. An optics assembly housing 174, for mounting the optics assembly 140, is disposed on the translating platform 164. (The illumination opening 167 and collection opening 168 are visible in FIG. 7; the imaging opening 169 is obscured by the optics assembly housing 174.)

The inside wall 170 of the delivery opening 166 may be coated with a reflective material, thereby minimizing loss of treatment light between the irradiator assembly 120 and the light guide 110. Alternatively, a reflector 171, with a reflective inner wall 172, may be disposed inside the delivery opening 166. The reflector 171 may be hollow or made of a solid transparent material.

FIG. 8 depicts a random ray-trace simulation of treatment light through the reflector 171 and light guide 110, with the apparatus 100 in the treatment mode. Treatment light is irradiated by the treatment light source 122, transferred by the reflector 171 and light guide 110 to the contact surface 112 of the light guide, then exiting the light guide 110 through the contact surface 112. Many of the rays may undergo one or more reflections within the reflector 171 and light guide 110. The irradiator assembly 120 may contain a treatment light filter 124; for example, a long pass optical filter or a band pass optical filter. The treatment light filter 124 may be interchangeable by the operator. The treatment light filter 124 is selected for providing a spectral irradiation in accordance with clinical indications.

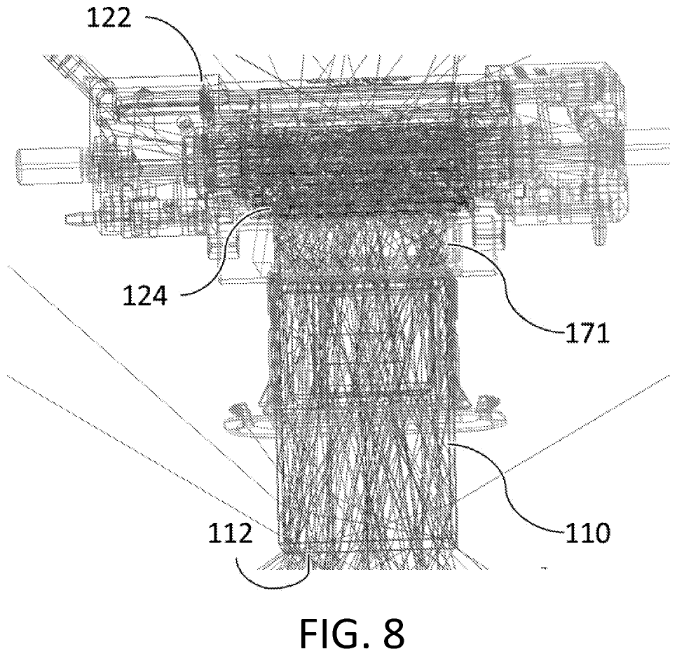

Reference is now made to FIGS. 9A-9B illustrating, in a block diagram, another non-limiting example of an apparatus 100A having a different configuration of an optical path selection assembly 160A configured to establish a treatment mode (shown in FIG. 9A) and a diagnostic mode (shown in FIG. 9B). It is noted that the apparatus 100A has same features and components as apparatus 100 except for what is described herein below. In this non-limiting example, the diagnostic system, which includes the illuminator assembly 130A, optics assembly 140A and imaging assembly 150A, is located side by side with the irradiator assembly 120A, above the light guide 110A. The optical path selection assembly 160A includes two adjacent mirrors 161A and 161B defining an optical path therebetween and are located above the light guide 110A. In the described non-limiting example, as shown in FIG. 9A, when in upright position, the mirrors establish a treatment optical path OP1 for the treatment light originating from the irradiator assembly 120A towards the light guide 110A that abuts the tissue. To establish a diagnostic optical path OP2, as further shown in FIG. 9B, the mirrors are moved/translated at their upper side, while their bottom side is kept in place above the light guide, such that they are inclined with a specific angle towards the output of the illuminator assembly and input of the imaging assembly 150A. In one example, the inclination angle is 45° to redirect the illumination light substantially perpendicular to the tissue, however this is not necessarily the case.

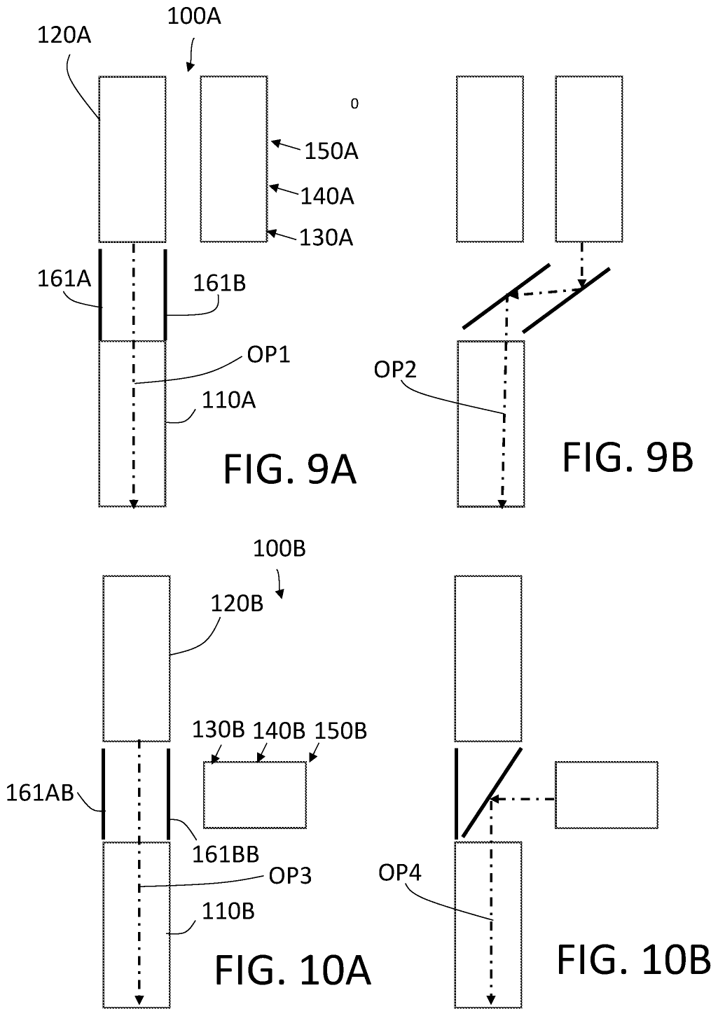

Reference is now made to FIGS. 10A-10B illustrating, in a block diagram, another non-limiting example of an apparatus 100B having a different configuration of an optical path selection assembly 160B configured to establish a treatment mode (shown in FIG. 10A) and a diagnostic mode (shown in FIG. 10B). It is noted that the apparatus 100B has same features and components as apparatus 100 except for what is described herein below. In this non-limiting example, the diagnostic system comprised of the illuminator assembly 130B, optics assembly 140B and imaging assembly 150B is located at a right angle with respect to the irradiator assembly 120B. The light guide 110B is on axis with the irradiator assembly 120B. It can be well perceived that this is only an example and the irradiator and diagnostic assemblies can switch their positions, and they can be oriented with a different angle therebetween. The optical path selection assembly 160B includes two adjacent mirrors 161AB and 161BB located above the light guide 110A. The mirror 161AB is kept stationary while the mirror 161BB has at least its bottom side translatable while its upper side can be fixed. In the described non-limiting example, as shown in FIG. 10A, when mirror 161BB is in upright position, the mirrors establish a treatment optical path OP3 for the treatment light originating from the irradiator assembly 120B towards the light guide 110B that abuts the tissue. Mirror 161BB blocks the diagnostic optical path. To establish a diagnostic optical path OP4, as further shown in FIG. 10B, the bottom of mirror 161BB is translated while its top side may be kept in place, such that the treatment optical path is blocked and the illumination light generated by the illuminator assembly can be directed towards the tissue after impinging on the inclined mirror 161BB. In the diagnostic mode, mirror 161AB does not participate. In one example, the inclination angle is 45° to redirect the illumination light substantially perpendicular to the tissue, however this is not necessarily the case.

Reference is made to FIG. 11, illustrating a non-limiting example of an apparatus 100C having a light guide 110C of a specific construction that enables treatment mode and diagnostic mode over two respective optical paths OP5 and OP6 through the light guide. this non-limiting example, enables activating the treatment and diagnosis modes without need for moving parts, e.g. without need for translatable optical path selection assembly. The optical path OP5 carrying the treatment light from the irradiator assembly 120C (located inside the apparatus) is substantially perpendicular to the treated tissue abutted by the contact surface 112C. The optical path OP6 carrying the illumination light and reflected/back-scattered light include is a broken path that inside the light guide propagates with an inclination such that the illumination and detection paths are angled with respect to the contact surface 112C and the abutted tissue. The illumination and detections paths may include one or more mirrors in between the illuminator and imaging assemblies 130C and 150C and the light guide 110C. In some embodiments, the treatment and diagnosis optical paths OP5 and OP6 may enable simultaneous activation of the treatment and diagnosis modes, in addition to serial/sequential activation thereof. When simultaneous activation is possible, the apparatus may include light barriers that stop treatment light from reaching the imaging assembly. The activation of the treatment and/or diagnosis mode is carried out by the controller.

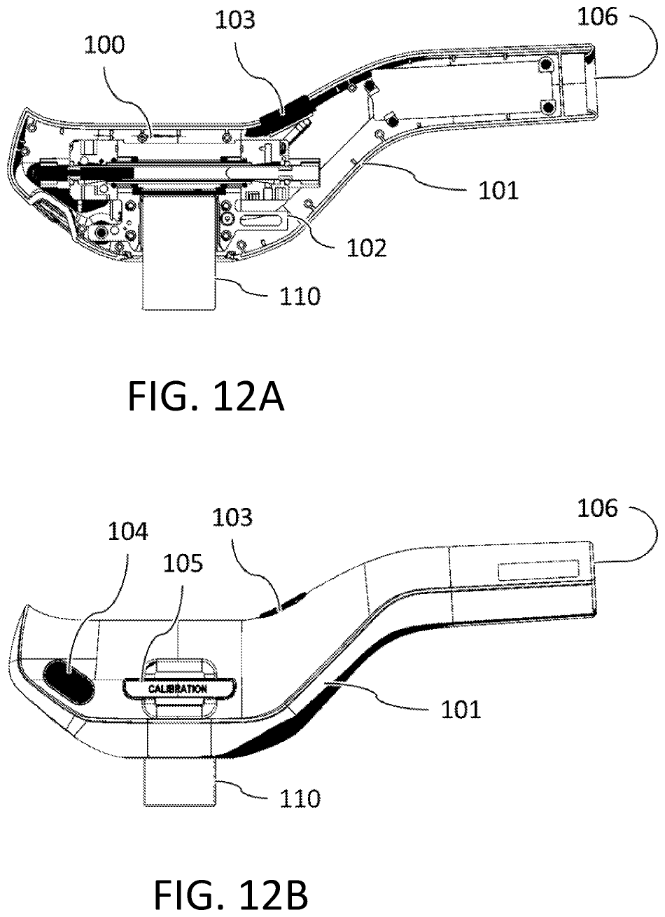

Reference is now made to FIGS. 12A-B. FIG. 12A depicts the apparatus 100 mounted in one of two half-shells of a handpiece housing 101 and FIG. 12B depicts a fully assembled handpiece 101, according to some non-limiting embodiments of the current disclosure. Typically, the handpiece 101 is connected to an external main unit (not shown) and enables an operator, with one hand, to position and apply the light guide 110 to a desired area of skin. The light guide 110 protrudes from the handpiece 101 to allow access to a patient's skin. A mounting bracket 102 may be used to secure the apparatus 100 in the handpiece 101. The handpiece 101 may be plastic, molded to fit and secure the apparatus 100 and the mounting bracket 102 when the half-shells of the handpiece 101 are assembled and fastened together. External treatment initiation buttons, such as a stem button 103 and/or a side button 104 may be placed on the handpiece 101 to control features of the apparatus 100. Two side buttons 104 may be placed on both sides of the handpiece and may have an identical function of initiating a treatment cycle. The operator may select which of the buttons to press according to their convenience, depending on how they are holding the handpiece 101. The treatment light filter 124, described above, is operator-insertable into the filter slot 105 located in the housing of the handpiece. A harness (not shown) may be connected at a connection end 106 of the handpiece 101, with appropriate strain relief at the connection end 106. The harness may carry electrical connections needed to operate and communicate with the apparatus 100, including connection to an external main unit (which may implement all or some functions, as described herein, of the controller of the apparatus 100). The harness may also circulate cooling water needed to cool the treatment light source 122 and/or the image sensor 152. The handpiece 101 may be ergonomically shaped for use by an operator performing skin treatment on the patient. The shapes and sizes of the handpiece 101 and light guide 110 may vary according to what section of skin is to be treated (face, arms, legs, torso, etc.).

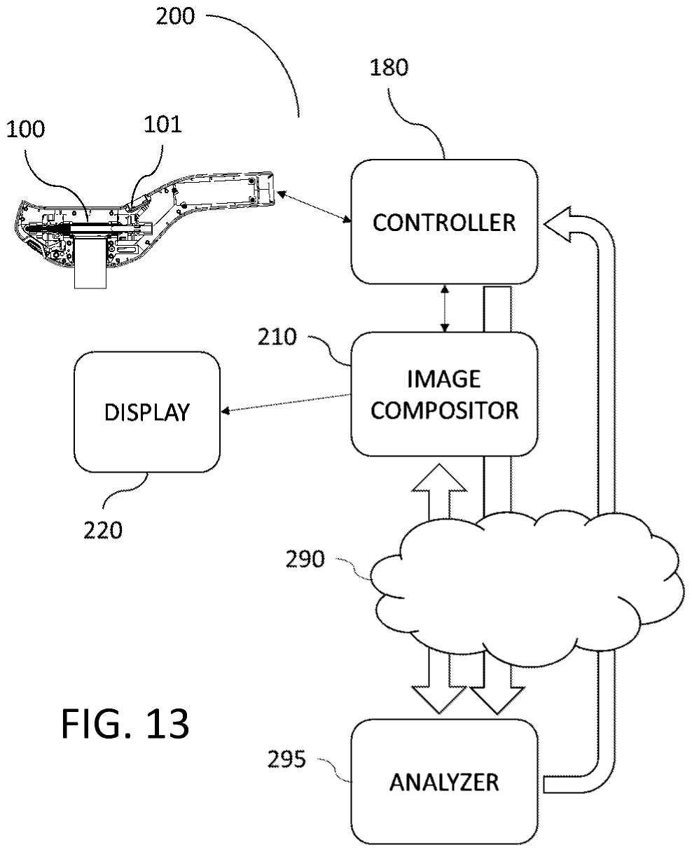

Reference is now made to FIG. 13, depicting a functional block diagram of a system 200 for skin imaging and diagnosis/analysis, according to some non-limiting embodiments. The system 200 comprises the apparatus 100, which may be housed in a handpiece 101, and a controller 180 that may be included in or external to the handpiece.

In some embodiments, the system 200 further comprises an image compositor 210. The image compositor 210 receives, from the controller 180, data of images acquired by the apparatus 100, taken during a diagnostic cycle of the apparatus 100. The images are of an abutted area of skin, taken under one or more different illumination spectra; for example, different images of the area of skin taken under illumination from LED's with different optical spectral outputs. The image compositor 210 may perform linear combinations of the different spectral images and/or perform non-linear operations on one or more of the spectral images, producing a composite image. The specific linear combination or algorithm of acquired spectral images depends on an image type, the image type defining which features of the skin are to be expressed in the composite image. (For example, the combination of spectral images chosen for a particular feature depends on the spectral contrast and the depth of the feature in the skin). Furthermore, a super composite image may be composed from primitive composite images. The image compositor 210 may furthermore identify, in an original or composite image, some features of the skin (e.g., hair, glands, blood vessels, etc.), which may be emphasized or subtracted in a descendant composite image. Composite images and/or skin parameters may be transmitted by the image compositor to the display 220, to be viewed by an operator of the apparatus 100.

In some embodiments, the image compositor 180 produces one or more of the following image types: an RGB image, a skin melanin map, a skin erythema map, a blood vessel map, a photon scattering map, an intermediate melanin map, a deep melanin map, a blood vessel depth map, tattoo ink analysis map, wrinkles map, lesion map, acne map, cellulite map, or any combination thereof.

In some embodiments, the system 200 further comprises an analyzer 295. The analyzer 295 may be remotely connected to the rest of the system 200 by a network 290. The analyzer 295 receives acquired images from the controller 180 and/or composited images from the image compositor 210. Based on the received images, the analyzer computes an optimal treatment course, at least for the next delivery, and sends the optimal treatment course, at least for the next delivery, to the controller 180 for implementation thereof, at least in the next cycle of treatment. Further details of the analyzer are provided elsewhere herein and in U.S. patent application Ser. No. 17/203,994, assigned to the assignee of this invention and incorporated herein by reference.

In some embodiments, the apparatus may transmit images to the analyzer and/or the image compositor, that compute numerical parameters of skin, based on the acquired and/or composite images. For example, the system may compute one or more of the following parameters:
1. A skin melanin level;
2. A skin erythema level;
3. A hair melanin level;
4. A hair diameter;
5. A hair density;
6. A hair width;
7. hair count;
8. blood vessel depth;
9. blood vessel diameter;
10. melanin contrast;
11. melanin depth; and
12. pigment depth.

It is understood that the functions of the controller 180, the image compositor 210, and/or the analyzer 295 may be implemented by any combination of software and in one or more pieces of hardware. Furthermore, the software and/or hardware may be disposed in proximity to the apparatus 100, remotely, or any combination thereof. Furthermore, the software and/or hardware may be accessible to the apparatus 100 by any short-, medium-, or long-distance networking means known in the art, wired or wirelessly.

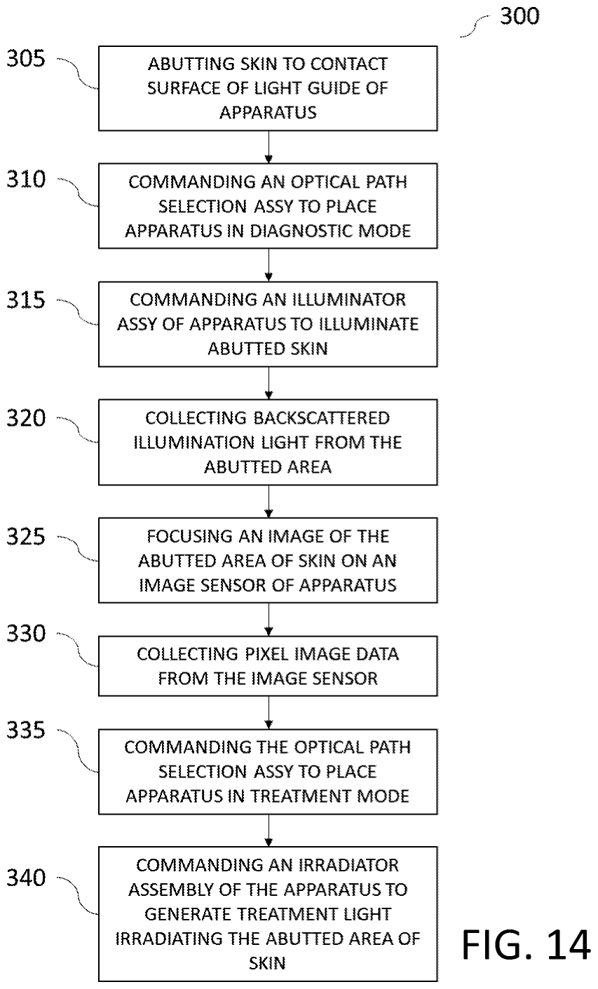

Reference is now made to FIG. 14 illustrating, by way of a flow chart, a method 300 for optical skin treatment procedures and in-treatment monitoring thereof, according to some embodiments of the invention.

The method 300 includes the following steps:
a. at 305, abutting a contact surface of a light guide of an apparatus, for optical skin treatment and in-treatment monitoring thereof, to an area of skin of a patient;
b. at 310, commanding, by a controller of the apparatus, an optical path selection assembly of the apparatus to place the apparatus in a diagnostic mode, whereby illumination and imaging paths are established;
c. at 315, commanding, by the controller, an illuminator assembly of the apparatus to illuminate the area of skin abutted to the contact surface;
d. at 320, collecting, by an optics assembly of the apparatus, backscattered illumination light from the abutted area of skin;
e. at 325, focusing, by the optics assembly, an image of the abutted area of skin on an image sensor of the apparatus;
f. at 330, commanding, by the controller, collection of image data of the image from the image sensor;
g. at 335, commanding, by the controller, the optical path selection assembly to place the apparatus in a treatment mode, whereby a treatment light delivery path is established; and
h. at 340, commanding, by the controller, an irradiator assembly of the apparatus to generate treatment light based on the analysis of the image data, and directing the treatment light through the light guide to irradiate the area of skin with the treatment light.

1. An apparatus for optical skin treatment and in-treatment monitoring thereof, comprising:
a controller;
a light guide comprising a contact surface configured for abutment to an area of skin;
an irradiator assembly configured, upon command of the controller, to provide treatment light to the abutted area of the skin;
an illuminator assembly configured, upon command of the controller, to provide illumination light to the abutted area of the skin;
an imaging assembly configured, upon command of the controller, to receive reflected or backscattered illumination light from the abutted area of the skin and generate skin images output data; and
an optical path selection assembly, configured to establish, upon command of the controller, a selected mode of the apparatus comprising either:
a treatment mode enabling the treatment light from the irradiator assembly to enter the light guide and irradiate the abutted area of skin to provide the skin treatment; or
a diagnostic mode, enabling the illumination light from the illuminator assembly to enter the light guide and illuminate the area of skin abutted to the contact surface; and the reflected or backscattered illumination light from the abutted area of the skin to traverse the light guide back to an image sensor of the imaging assembly;
the controller further configured to:
analyze the reflected or backscattered illumination light from the abutted area of the skin;
provide a treatment light regimen, based on the analyzed reflected or backscattered illumination light;
select a treatment mode; and
activate the treatment light according to the treatment regimen, thereby treating the skin.

2. The apparatus of claim 1, further comprising an optics assembly configured to collect the reflected or backscattered illumination light and direct it towards the imaging assembly.

3. The apparatus of claim 1, wherein the irradiator assembly comprises an extended light source and one or more reflectors between the extended light source and the light guide, the reflector being configured to capture and reflect rays of the extended light source towards the light guide.

4. (canceled)

5. The apparatus of claim 1, wherein said illuminator assembly comprises an illumination polarizer configured so that said illumination light is P-polarized in a plane of incidence of the illumination light.

6. The apparatus of claim 5, wherein said imaging assembly comprises a detection polarizer, disposed upstream the image sensor and arranged cross-polarized with the illumination polarizer.

7. (canceled)

8. The apparatus of claim 1, wherein the illuminator assembly comprises plurality of sets of one or more light sources, each set having a different illumination spectrum, and the controller is further configured to select illumination from one or more of the sets of light sources.

9-11. (canceled)

12. The apparatus of claim 1, wherein the imaging assembly comprises a position adjustment mechanism to adjust position of the image sensor relative to a plane of the image sensor.

13. The apparatus of claim 1, wherein the optical path selection assembly comprises a translatable platform, the platform being translatable on a stationary frame of the apparatus between two positions to establish said treatment and diagnostic modes respectively, wherein the irradiator assembly and the light guide are in rigid connection with the stationary frame.

14. The apparatus of claim 13, wherein the optics assembly comprises a first folding mirror, a lens assembly, and a second folding mirror, wherein at least the illuminator assembly and the first folding mirror are rigidly affixed to the translatable platform.

15-19. (canceled)

20. The apparatus of claim 1, wherein the optical path selection assembly comprises a pair of movable mirrors configured to be moved between at least two positions to said treatment and diagnostic modes respectively.

21. The apparatus of claim 1, wherein the optical path selection assembly comprises a single movable mirror configured to be moved between at least two positions to said treatment and diagnostic modes respectively.

22. The apparatus of claim 1, wherein the optical path selection assembly comprises two light input surfaces configured to switch between said treatment and diagnostic modes.

23. (canceled)

24. The apparatus of claim 8, further comprising an image compositor, enabled to acquire pixel data of the images taken under one or more of the different illumination spectra and apply linear mixing and/or algorithms to produce one or more composite images.

25. The apparatus of claim 24, wherein the image compositor is configured to produce one or more of the following types of composite images: an RGB image, a melanin map, an erythema level map, a blood vessel map, a photon scattering map, an intermediate melanin map; a deep melanin map, and a blood vessel depth map.

26. A method for optical imaging and cosmetic treatment of skin, comprising:
abutting a contact surface of a light guide of an apparatus to an area of the skin of a patient for optical skin treatment and in-treatment monitoring thereof;
commanding, by a controller of the apparatus, an optical path selection assembly of the apparatus to place the apparatus in a diagnostic mode, whereby illumination and imaging paths are established;
commanding, by the controller, an illuminator assembly of the apparatus to illuminate the area of skin abutted to the contact surface;
collecting, by an optics assembly of the apparatus, backscattered illumination light from the abutted area of skin;
commanding, by the controller, collection of image data of a plurality of images from the image sensor;
analyzing, by the controller, the image data;
generating, by the controller, treatment light parameters based on the analyzed image data;
commanding, by the controller, the optical path selection assembly to place the apparatus in a treatment mode, whereby a treatment light delivery path is established;
commanding, by the controller, an irradiator assembly of the apparatus to generate treatment light having parameters based on the analysis of the image data; and
directing the treatment light through the light guide to irradiate the area of abutted skin with the treatment light.

27. (canceled)

28. A system for optical skin treatment and in-treatment monitoring thereof, comprising:
i) an energy treatment handpiece comprising an irradiator assembly configured to provide treatment light; a light guide having a contact surface configured for abutment to an area of skin; and an optical path configured to pass the treatment light from the irradiator assembly to enter the light guide and irradiate the abutted area of skin to provide the skin treatment to the abutted area of skin;
ii) a skin imaging handpiece comprising;
an illuminator assembly having a plurality of light sources configured to provide illumination light of different wavelengths of light to the skin;
an imaging assembly having one or more sensors configured to receive reflected measured light; and
one optical path configured to pass illumination light from the illuminator assembly to the skin and pass the reflected or backscattered illumination light back to one or more sensors of the imaging assembly;
iii) a controller, the controller configured to:
illuminate by the illuminator assembly of the skin imaging handpiece the skin;
receive, from an output of the one or more sensors, measured light information of light being reflected from the skin illuminated by the illuminator assembly wherein the measured light information comprises information of different depths in the skin tissue based on illuminating the skin tissue with the different wavelengths of light;
analyze the measured light information; and
provide a treatment light regimen, based on the measured light information.

29. The system of claim 28, wherein the energy treatment handpiece is placed in position to treat the skin and activated, by the controller, to provide the treatment light according to the treatment regimen, thereby treating the abutted area of the skin.

30. The system of claim 28, wherein the controller is configured to employ artificial intelligence and deep learning methods contained in a memory associated with the programmable controller to analyze the measured light information and select the treatment light regimen.

31. A method for optical skin treatment and in-treatment monitoring thereof, comprising:
providing an energy treatment handpiece comprising an irradiator assembly configured to provide treatment light; a light guide having a contact surface configured for abutment to an area of skin; and an optical path configured to pass the treatment light from the irradiator assembly to enter the light guide and irradiate the abutted area of skin to provide the skin treatment to the abutted area of the skin;
providing a skin imaging handpiece comprising;
an illuminator assembly having a plurality of light sources configured to provide illumination light of different wavelengths of light to the skin;
an imaging assembly having one or more sensors configured to receive measured light reflected from the abutted are of the skin; and
one optical path configured to pass illumination light from the illuminator assembly to the skin and pass reflected or backscattered illumination light from the abutted are of the skin to one or more sensors of the imaging assembly;
providing a controller;
placing the skin imaging handpiece in a position to image the skin;
illuminating, by the controller and the illuminator assembly, the skin imaging handpiece the skin;
receiving, by the controller, from an output of the one or more sensors, measured light information of light being reflected from the skin tissue illuminated by the illuminator assembly, wherein the measured light information comprises information of different depths in the skin tissue based on illuminating the skin tissue with the different wavelengths of light;
analyzing, by the controller, the measured light information;
providing, by the controller, a treatment light regimen, based on the measured light information.

32. The method of claim 31, the method further comprising:
placing the treatment handpiece in position to treat the skin; and
activating, by the controller, the energy treatment handpiece to provide the treatment light according to the treatment regimen, thereby
treating the skin tissue.

33. The method of claim 31, wherein the controller is configured to employ artificial intelligence and deep learning methods contained in a memory associated with the programmable controller to analyze the measured light information and select the treatment light regimen.
